(19) United States
(12) Patent Application Publication
(10) Pub. No.: US 2009/0164243 A1
(43) Pub. Date: Jun. 25, 2009

(54) ELECTRONIC HEALTHCARE IDENTIFICATION GENERATION AND RECONCILIATION

(75) Inventors: John A. Zubak, Doylestown, PA (US); Harvey Mitgang, Princeton Junction, NJ (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: J&H Enterprises, LLC, Wilmington, DE (US)

(21) Appl. No.: 12/362,092

(22) Filed: Jan. 29, 2009

Related U.S. Application Data
(63) Continuation-in-part of application No. 11/240,872, filed on Sep. 30, 2005.
Application No. 11/240,872 is a continuation-in-part of application No. 12/360,657, filed on Jan. 27, 2009.

(51) Int. Cl. G06Q 50/00; G06Q 40/00; G06Q 90/00

(52) U.S. Cl. 705/2; 705/4

(57) ABSTRACT

A system and method for generating healthcare identification and reconciliation information are provided. In an illustrative implementation, a computer-implemented healthcare information and reconciliation platform (HIRP) maintains a HIR engine which is operable on various data including but not limited to patient data, network data, plan data, insurance carrier/payor data, and healthcare provider data. In an illustrative operation, the HIR engine can receive input data representative of a participating user's benefit plan. The HIR engine can process the inputted data and correlate the inputted data against healthcare provider data, benefit plan data, healthcare related data, healthcare network data and insurance carrier/payor data to generate and/or store an electronic healthcare card/document representative of the most up-to-date contractual obligations between the cooperating parties (e.g., healthcare provider, benefit plan administrator, healthcare network, insurance carrier, and the covered person/patient).

### CROSS REFERENCE TO RELATED APPLICATIONS AND CLAIM FOR PRIORITY

The present application claims priority to and is a continuation-in-part of U.S. patent application Ser. No. 11/240,872, filed on Sep. 30, 2005, entitled, “Electronic Healthcare Identification and Reconciliation,” the contents of which are hereby incorporated by reference in its entirety. The present application also claims priority to and is a continuation-in-part of U.S. patent application Ser. No. 12/360,657, filed on Jan. 27, 2009, entitled, “Electronic Healthcare Identification and Reconciliation,” the contents of which are hereby incorporated by reference in its entirety.

The present application is further related by subject matter to the following applications, the contents of which are hereby incorporated by reference: U.S. patent application Ser. No. 11/400,929, filed on Apr. 10, 1996, and entitled “Electronic Healthcare Identification Generation and Management;” U.S. patent application Ser. No. 11/805,443, filed on May 23, 2007, and entitled, “Electronic Healthcare Information Management For On-Demand Healthcare;” U.S. patent application Ser. No. 11/903,087, filed on Sep. 20, 2007, and entitled “Management of Healthcare Information in a Quilted Healthcare Network;” and U.S. patent application Ser. No. 12/059,207, filed on Mar. 31, 2008, and entitled “Hybrid Healthcare Identification Platform.”

### BACKGROUND

The management of healthcare information can be arduous and time consuming. More importantly, ineffective management of healthcare information can be costly to healthcare providers, patients, and insurance companies/payors alike. Current healthcare practices rely on managed healthcare systems that create relationships between healthcare providers, insurance companies/payors, and patients. These include various types of medical access such as traditional health benefits, workers compensation medical treatment and others. In this context, patients and or employers generally maintain a medical plan provided by an insurance carrier or, in increasing frequency, self insuring and/or participating in specialty programs outside of the traditional employer-provided insurance environment. The method of access to the medical benefits that a particular plan, insured, and/or patient can choose that provides financial coverage and that minimizes out-of-pocket expenses can contain various rules, regulations, and restrictions. Such rules, regulations, and restrictions can include but are not limited to the frequency of healthcare provider visits, which healthcare providers can be seen, which “network” (e.g., approved healthcare providers that have established relationships with the medical benefit/health insurance plan), which prescriptions are covered by the health insurance plan, if any, and other contractual requirements and restrictions that must be fulfilled to assure that the cost of the medical services are covered by the medical benefit plan so that the cost to payors (e.g., an insurance carrier, plan administrator, etc.) is minimized.

A medical benefit/health insurance plan is generally provided by an insurance carrier to one or more insured parties. The medical benefit/health insurance plan can operate to establish relationships with private healthcare providers such that price certainty is achieved for particular healthcare services provided by the healthcare service providers. The healthcare providers who engage in such relationships are generally considered to be part of a “network” of healthcare providers. The distinction of being in “network” and out of “network” is important to the payors and the insured or covered party (e.g., patient) as, generally, in “network” healthcare providers have contractual relationships which if utilized by the covered person translates into less expense for the payors.

Given increasing competition between medical benefit plans, the proper utilization of contractual agreements between providers, networks and payors is imperative to control the costs of the plans. Although, such arrangement is beneficial primarily the payors and healthcare providers, all of the parties including the insured parties/covered persons can be left exposed to a scenario where a trusted healthcare provider is in “network” one day and then out of “network” another day as the contractual agreements between the various parties change. In such context, the payors, insured parties and other covered persons can be exposed to higher expenses if the covered person continues to see the healthcare provider without compliance to the established contractual requirements. With current practices, it is often the case that the covered person does not realize the contractual requirements and/or the change in “network” designation until they receive a bill for services indicating to the covered person that were either not covered or only partially covered as a result of non-compliance to the established contractual requirements.

Further, given increasing choices between medical plans, healthcare providers and payors are left to perform arduous back office processing when reconciling payments for covered persons. For example, a healthcare provider might subscribe to three different healthcare networks (e.g., Network A, Network B, and Network C). However, the covered person's benefit plan might only contractually be eligible for Network B. Without proper compliance by the covered person and the benefit plan to Network B's contractual requirements, the cost savings related to the services provided by the healthcare provider could be lost. In certain contexts, the healthcare provider can be made privy to particular coverage by the instructions and/or identifying logo on the covered person's healthcare identification card. Such logos are an example of what can be contractually required by healthcare providers to be present on the covered party's healthcare identification card as a condition for the healthcare provider to accept discounted payment for services provided.

With current practices, however, given the costs associated with the production and distribution of healthcare identification cards, insurance carriers often issue one healthcare identification card annually to the covered party. With current practices, the healthcare identification card does not accurately reflect the benefits afforded to the covered party as such benefits often change during the course of a year. More importantly, with current practices, network access requirements such as required logos (that can change during the covered party's coverage period) might not be present on the annually distributed healthcare identification cards leaving payors responsible to pay non-discounted prices to healthcare service providers for services rendered. In this context, the covered persons are also exposed to increased costs as payors will, in some cases, pass on their increased costs to their insured parties either directly or in the form of increased insurance plan costs/premiums.

Moreover, with current practices, participating users (e.g., insured parties) are relegated to searching for various healthcare information at differing sources. For example, an employee can enroll for healthcare insurance as provided by his/her employer. Additionally, the employee can appoint a certain part of their paycheck to be saved in a tax deferred savings account. With current practices, in this example, the participating user would have to search for his/her healthcare insurance information (e.g., benefit restrictions, in-network doctors, co-pay information desired procedure, etc.) from a source associated with the healthcare insurance provider and at a second source to determine how much he/she has in their healthcare spending account. The current lack of aggregation of inter-related healthcare information renders its management, at best, an arduous and cumbersome task by its consumers that include patients, healthcare service providers, insurance providers, healthcare billing and payment parties, and employers.

Further, with current practices, healthcare identification (and other information) is not easily tracked, stored, and or monitored from a central location. Since, typically, such information is not centrally managed, stored, tracked and/or monitored, the task of generating reports using various components of this information (e.g., tracking and/or monitoring the usage of specific healthcare services) can be arduous and difficult. The difficulty in generating such reports (and/or tracking such healthcare related activities) can result in increased healthcare costs. For example, without such information, healthcare insurance providers, healthcare plan providers, workman's compensation providers, benefits administrators and the like cannot identify and manage healthcare claims and cannot provide guidance to patients regarding treatment options that might possibly avert unneeded or cumulative healthcare service costs.

From the foregoing, it is appreciated that there exists a need for systems and methods that provide updated, real-time electronic healthcare identification and reconciliation information aimed to ameliorate the shortcomings of existing practices.

### SUMMARY

The herein described systems and methods provide a computer-implemented interactive system and methods for generating healthcare identification and reconciliation information. In an illustrative implementation, a healthcare information and reconciliation platform (HIRP) comprises a HIR engine operating on a plurality of patient, healthcare provider, plan, and insurance carrier/payor data, and a graphical user interface operable to receive input data and display data representative of an electronic healthcare identification card. In the illustrative implementation, the plurality of patient, healthcare provider, plan, and insurance carrier/payor data is updated on a selected time interval (e.g., daily).

In an illustrative implementation, a participating user (a subscriber/covered person) and/or healthcare provider personnel (an administrative clerk, admitting nurse, receptionist, etc.) can input data representative of the participating subscriber's (e.g., covered person) medical benefit plan (e.g., patient identification number, insurance plan number, plan member number, provider, etc.) to the HIR engine through an electronic communications interface which may comprise, for example, an exemplary graphical user interface, an interactive telephone interface, a customer service telephone number, and/or an electronic messaging system such as email or text messaging. Responsive to the inputted data, the HIR engine can operate to process the input data and correlate the inputted data with healthcare provider data, plan data and insurance carrier/payor data to generate an electronic healthcare card (i.e., which can then be printed) which contains thereon data required to satisfy contractual obligations that exist between the insurance carrier/payors and health care service provider (e.g., placement of selected logos on the electronic healthcare card/document which are required by contract between the healthcare service provider, managed care networks, and the insurance carrier/payors so that the healthcare service provider accepts a discounted fee from the insurance carrier/payor for services provided to the covered person—i.e., patient/subscriber).

In the illustrative operation, the correlation processing can identify if the participating subscriber is eligible to select a set or subset of healthcare providers for use in obtaining healthcare services. The eligibility determination can be realized by comparing the inputted data from the participating user against selected requirements set forth in plan designs and explanations of benefits provided by the plan sponsor/insurance carrier/payor and identifying restrictions/requirements present in service contracts that exist between the parties. In the event an eligibility check is being performed by a healthcare service provider or someone other than the subscriber as discussed in connection with FIG. 5A below, it may not be necessary to confirm eligibility to select a specific provider in order to move forward with accessing the system. Indeed, the specific steps employed to access the system, and the particular eligibility processing that is performed, may vary depending upon the entity or person that is accessing the system as well as the particular information that the entity or person is seeking to obtain.

Further in the illustrative operation, the correlation processing can be used to generate the illustrative electronic healthcare card/document which can be indicative of various most-up-to-date (e.g., current) healthcare information and related healthcare information for the subscriber (and other cooperating parties) including but not limited to the contract obligations the healthcare service providers are performing under at a selected time period, which discounts are being offered between the insurance carrier/payors and the healthcare service provider, which contractual obligations must be met for the discounts to take effect (e.g., placement of selected logos on the electronic healthcare card), remaining deductible amount available to the participating user, health savings account balances and updates, indemnity plan details (e.g., indemnity schedules, tables, and data), instructions to HMOs and other benefit plan providers to facilitate a specific plan's requirements, and co-pay information for the participating subscriber.

In the illustrative implementation, the electronic healthcare card/document can be generated, stored, and displayed on the graphical user interface operating in an illustrative computing environment, communicated via telephone to a participating user, faxed and/or emailed to a healthcare service provider, displayed on a mobile phone or other mobile communication device having a display area, and can also be printed for presentation to a healthcare service provider. In the illustrative operation, the healthcare provider can use the information from electronic healthcare card/document, which may be stored, communicated, displayed, and/or printed, as part of payment reconciliation processing performed between the healthcare provider and the insurance carrier/payor.

In the illustrative operation, the exemplary HIR engine can provide various electronic links to one or more cooperating data stores having data representative of healthcare forms (e.g., specialty forms) for use in processing claims under a selected benefit plan. In the illustrative operation, a participating user may be provided forms by the exemplary HIR engine based on the occurrence of one or more selected events (e.g., participating user wishing to assign a benefit to a particular healthcare service provider). Further in the illustrative operation, HIRP can operate to identify specific vendor partners that have contracted with payors and provide direction and steerage (e.g., of participating users using the HIRP) to such partners.

In the illustrative operation, generated healthcare card/documents can be stored for use by one or more cooperating parties. In the illustrative operation, the generated healthcare card/documents can be used in a selected data mining operation to determine, monitor, and/or track various activities including but not limited to utilization of healthcare card/documents, assignment of benefits, utilization of particular healthcare service providers, etc.

Other features of the herein described systems and methods are further described below.

### BRIEF DESCRIPTION OF THE DRAWINGS

The interactive systems and methods for generating electronic healthcare identification and reconciliation information are further described with reference to the accompanying drawings in which:

FIG. 1 is a block diagram of an exemplary computing environment in accordance with an implementation of the herein described systems and methods;

FIG. 2 is a block diagram showing the cooperation of exemplary components of an illustrative implementation in accordance with the herein described systems and methods;

FIG. 3 is a block diagram showing the cooperation of exemplary components of another illustrative implementation in accordance with the herein described systems and methods;

FIG. 4 is a block diagram showing an illustrative block representation of an illustrative healthcare identification and reconciliation system in accordance with the herein described systems and methods;

FIG. 5 is flow diagram showing illustrative processing performed when generating healthcare identification and reconciliation information in accordance with the herein described systems and methods;

FIG. 5A is a flow diagram showing other illustrative processing performed when generating healthcare identification and reconciliation information in accordance with the herein described systems and methods;

FIG. 6 is flow diagram showing another illustrative processing performed when generating healthcare identification and reconciliation information in accordance with the herein described systems and methods;

FIG. 7 is flow diagram showing another illustrative processing performed when generating healthcare identification and reconciliation information in accordance with the herein described systems and methods;

FIG. 8 is flow diagram showing another illustrative processing performed when generating healthcare identification and reconciliation information in accordance with the herein described systems and methods;

FIG. 9 is flow diagram showing another illustrative processing performed when generating healthcare identification and reconciliation information in accordance with the herein described systems and methods;

FIG. 10 is flow diagram showing another illustrative processing performed when generating healthcare identification and reconciliation information in accordance with the herein described systems and methods; and

FIG. 11 is flow diagram showing another illustrative processing performed when generating healthcare identification and reconciliation information in accordance with the herein described systems and methods.

### DETAILED DESCRIPTION

### Illustrative Computing Environment

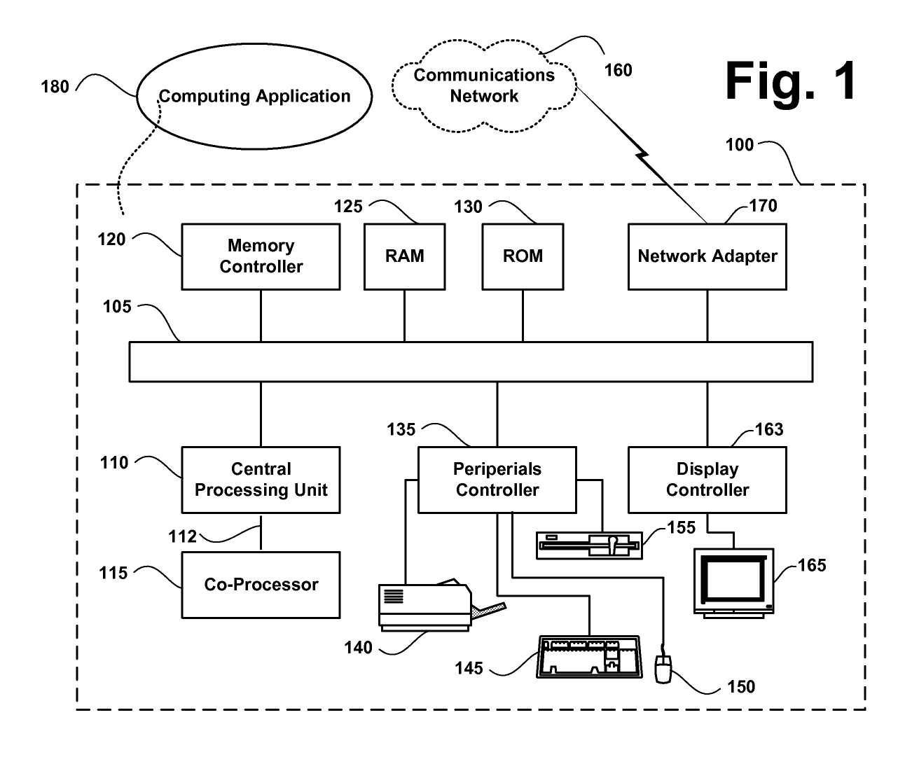

FIG. 1 depicts an exemplary computing system 100 in accordance with herein described system and methods. The computing system 100 is capable of executing a variety of computing applications 180. Computing application 180 can comprise a computing application, a computing applet, a computing program and other instruction set operative on computing system 100 to perform at least one function, operation, and/or procedure. Exemplary computing system 100 is controlled primarily by computer readable instructions, which may be in the form of software. The computer readable instructions can contain instructions for computing system 100 for storing and accessing the computer readable instructions themselves. Such software may be executed within central processing unit (CPU) 110 to cause the computing system 100 to do work. In many known computer servers, workstations and personal computers CPU 110 is implemented by micro-electronic chips CPUs called microprocessors. A coprocessor 115 is an optional processor, distinct from the main CPU 110 that performs additional functions or assists the CPU 110. The CPU 110 may be connected to co-processor 115 through interconnect 112. One common type of coprocessor is the floating-point coprocessor, also called a numeric or math coprocessor, which is designed to perform numeric calculations faster and better than the general-purpose CPU 110.

In operation, the CPU 110 fetches, decodes, and executes instructions, and transfers information to and from other resources via the computer's main data-transfer path, system bus 105. Such a system bus connects the components in the computing system 100 and defines the medium for data exchange. Memory devices coupled to the system bus 105 include random access memory (RAM) 125 and read only memory (ROM) 130. Such memories include circuitry that allows information to be stored and retrieved. The ROMs 130 generally contain stored data that cannot be modified. Data stored in the RAM 125 can be read or changed by CPU 110 or other hardware devices. Access to the RAM 125 and/or ROM 130 may be controlled by memory controller 120. The memory controller 120 may provide an address translation function that translates virtual addresses into physical addresses as instructions are executed.

In addition, the computing system 100 can contain peripherals controller 135 responsible for communicating instructions from the CPU 110 to peripherals, such as, printer 140, keyboard 145, mouse 150, and data storage drive 155. Display 165, which is controlled by a display controller 163, is used to display visual output generated by the computing system 100. Such visual output may include text, graphics, animated graphics, and video. The display controller 163 includes electronic components required to generate a video signal that is sent to display 165. Further, the computing system 100 can contain network adaptor 170 which may be used to connect the computing system 100 to an external communication network 160.

### Illustrative Network Computing Environment:

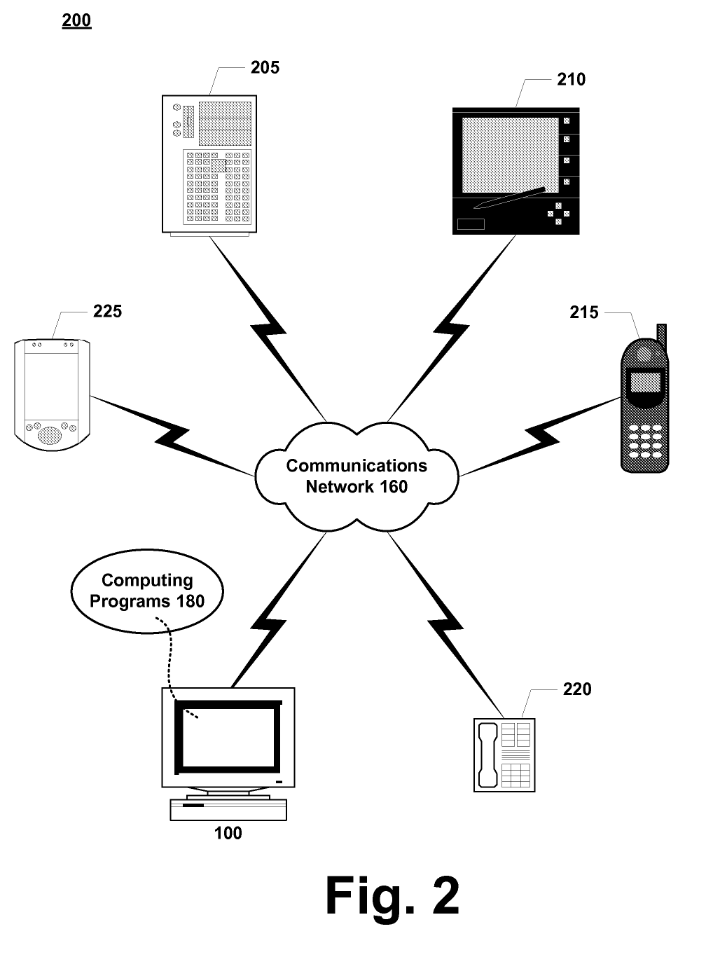

Computing system 100, described above, can be deployed as part of a computer network. In general, the above description for computing environments applies to both server computers and client computers deployed in a network environment. FIG. 2 illustrates an exemplary illustrative networked computing environment 200, with a server in communication with client computers via a communications network, in which the herein described apparatus and methods may be employed. As shown in FIG. 2, server 205 may be interconnected via a communications network 160 (which may be either of, or a combination of a fixed-wire or wireless LAN, WAN, intranet, extranet, peer-to-peer network, virtual private network, the Internet, or other communications network) with a number of client computing environments such as tablet personal computer 210, mobile telephone 215, telephone 220, personal computer 100, and personal digital assistance 225. In a network environment in which the communications network 160 is the Internet, for example, server 205 can be dedicated computing environment servers operable to process and communicate data to and from client computing environments 100, 210, 215, 220, and 225 via any of a number of known protocols, such as, hypertext transfer protocol (HTTP), file transfer protocol (FTP), simple object access protocol (SOAP), or wireless application protocol (WAP). Additionally, networked computing environment 200 can utilize various data security protocols such as secured socket layer (SSL) or pretty good privacy (PGP). Each client computing environment 100, 210, 215, 220, and 225 can be equipped with operating system 180 operable to support one or more computing applications, such as a web browser (not shown), or other graphical user interface (not shown), or a mobile desktop environment (not shown) to gain access to server computing environment 205.

In operation, a user (not shown) may interact with a computing application running on a client computing environments to obtain desired data and/or computing applications. The data and/or computing applications may be stored on server computing environment 205 and communicated to cooperating users through client computing environments 100, 210, 215, 220, and 225, over exemplary communications network 160. A participating user may request access to specific data and applications housed in whole or in part on server computing environment 205. These data may be communicated between client computing environments 100, 210, 215, 220, and 220 and server computing environments for processing and storage. Server computing environment 205 may host computing applications, processes and applets for the generation, authentication, encryption, and communication of web services and may cooperate with other server computing environments (not shown), third party service providers (not shown), network attached storage (NAS) and storage area networks (SAN) to realize such web services transactions.

### Healthcare Identification and Reconciliation:

### Overview:

The herein described systems and methods provide for the generation of an electronic healthcare card/document that can be generated specific to each covered party, plan and, healthcare provider on a real time electronic basis versus a traditional hard copy identification card. In illustrative implementations, the electronic healthcare card/document can be used for various medical benefit programs including but not limited to health, workers compensation, occupational accident, and student accident polices.

In an illustrative operation, an insured or covered party can be provided with a provider directory website in which they can locate a provider within their insurance plan. Upon locating a provider, the covered party can then be prompted to generate an electronic healthcare card/document. Such prompts can also include one or more requests to the covered party to input into an illustrative healthcare identification and reconciliation platform relevant information specific to the covered party's particular plan. In the illustrative operation, the exemplary healthcare identification and reconciliation platform can verify the inputted information and generate the requested electronic healthcare card/document which among other things can contain information representative of the information required by the various parties related to the benefit plan. Such information can be required to satisfy one or more contractual obligations that exist between the insurance carrier/payor and one or more healthcare (service) providers (e.g., the presence of one or more selected logos on the electronic healthcare card which are required by the healthcare provider as a condition of accepting discounted payment for services rendered).

In the illustrative operation, once the electronic healthcare card is generated, the covered party can utilize the electronic healthcare card/document in the same manner as a traditional healthcare identification card. Stated differently, the information a healthcare provider requires to verify insurance coverage and requires to identify network participation can be readily available to both the covered party and the healthcare provider.

In using the electronic healthcare card/document the production of traditional (e.g., annually distributed) healthcare identification cards can be eliminated which can result in a reduction in overhead costs for the insurance carrier/payor (or the employer group should an employer act as the source of insurance to an covered party). Further, the electronic healthcare card of the herein described systems and methods can operate to ensure that the most up-to-date (e.g., current) benefit plan information is presented to the healthcare provider and is deployed by the insurance carrier/payor to the covered party.

In the illustrative operation, once a covered party is verified as an eligible member of a given benefit plan, a healthcare provider/network can be assigned by the plan and/or can be chosen by the covered party using an exemplary healthcare identification and reconciliation platform in accordance with the herein described systems and methods. In such context, the healthcare identification and reconciliation platform can operate to provide a list of healthcare providers to the covered party that are available within a given benefit plan. Such operation can be realized by correlating information inputted to the healthcare identification platform by the covered party with healthcare provider information, healthcare network, insurance carrier/payor and plan design information. The desired healthcare provider selected, the healthcare identification and reconciliation platform can operate to generate an electronic healthcare card/document that can be specific to the selected plan and healthcare provider containing appropriate personal plan option (PPO), healthcare management organization (HMO), and/or managed care network information necessary for proper selection of the services.

With current practices, in various instances, given the size of traditional healthcare identification cards and amount of information that is required to be provided to healthcare providers, insurance carriers/payors are detrimentally positioned to leave vital information off of the traditional healthcare identification card. In such instances, when healthcare providers are contacted by the insured/covered party to make an appointment with the healthcare provider using traditional healthcare identification cards, important information can be missing from the traditional healthcare identification card which can lead to unnecessary out-of-pocket expenses to be paid by the responsible party. Specifically, by using information from deficient traditional healthcare identification cards, it can be difficult for a healthcare provider to confirm to the covered party whether the healthcare provider is a participating healthcare provider (e.g., in “network”) in the insured/covered party's benefit plan. Consequently, healthcare providers, in such instance, can require the covered party to pay full-billed charges directly to the healthcare provider and require the covered party to submit the healthcare provider bill for services rendered directly to the insured/covered party's insurance carrier/payor (or employer) for reimbursement.

The electronic healthcare card/document of the herein described systems and methods aims to ameliorate the shortcomings of existing practices by providing real-time, up-to-date (current) healthcare identification information (e.g., insurance, payor or provider information) to the healthcare provider that allows healthcare providers to identify the healthcare provider's participation in the covered party's benefit plan such that the insured/covered party's payor does not have to incur any undue costs. Additionally, in an illustrative embodiment, the disclosed systems are adapted to provide supplemental information related to the specific coverage or benefit plan and communicate potentially relevant information to cooperating parties. Further, in an illustrative embodiment, the disclosed systems may be adapted to provide electronic links to additional healthcare management information (e.g., forms required for healthcare reconciliation processing) that may be related to the benefit coverage provided by the benefit plans for intended use by participating users (e.g., insured parties).

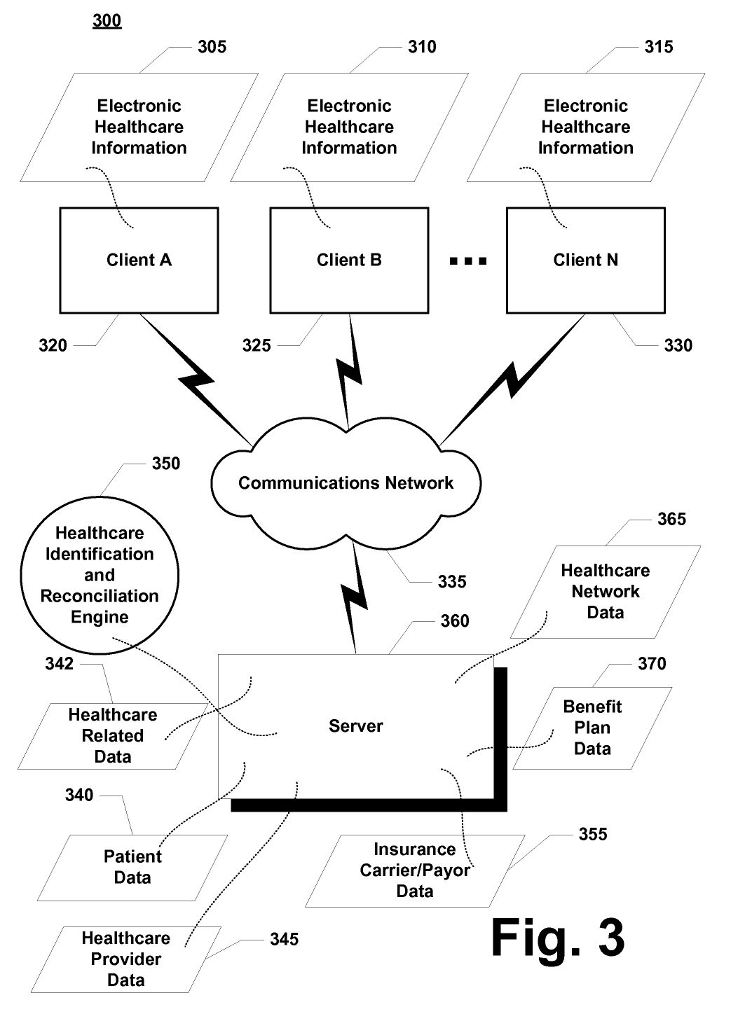

FIG. 3 shows an illustrative implementation of exemplary healthcare identification and reconciliation platform 300. As is shown in FIG. 3, exemplary healthcare identification and reconciliation platform 300 comprises client computing environment 320, client computing environment 325 up to and including client computing environment 330, communications network 335, server computing environment 360, healthcare identification and reconciliation engine 350, patient data 340, healthcare provider data 345, insurance carrier/payor data 355, healthcare network data 365, and benefit plan data 370. Also, as is shown in FIG. 3, healthcare identification and reconciliation platform can comprise a plurality of electronic healthcare cards/documents 305, 310, and 315 which can be displayed, viewed, electronically transmitted and printed from client computing environments 320, 325, and 330, respectively.

In an illustrative operation, client computing environments 320, 325, and 330 can communicate with server computing environment 360 over communications network 335 to provide requests for and receive electronic healthcare information 305, 310, and 315. In the illustrative operation, healthcare identification and reconciliation engine 350 can operate on server computing environment 360 to provide one or more instructions to server computing environment 360 to process requests for healthcare information 305, 310, and 315 and to provide healthcare information 305, 310, and 315 to the requesting client computing environment (e.g., client computing environment 320, client computing environment 325, or client computing environment 335). As part of processing requests for healthcare identification and reconciliation card/document information 305, 310, and 315, healthcare identification and reconciliation engine 350 can utilize a plurality of data including patient data 340, healthcare provider data 345, healthcare related data 342, insurance carrier/payor data 355, healthcare network data 365, and benefit plan data 370. Also, as is shown in FIG. 3, client computing environments 320, 325, and 330 are capable of processing healthcare identification and reconciliation card/document information 305, 310, and 315 for display and interaction to one or more participating users (not shown).

Currently, insured or covered persons and/or healthcare providers may access a plurality of different sources in an attempt to clarify the covered person's benefits. For example, healthcare providers might access a plurality of resources (e.g., pamphlets, etc) and different systems such as, for example, a web-based portal supported by the insurance company, in order to piece together patient eligibility and related healthcare information. Often the information is disjointed and is received by participating users (e.g., insured parties) in various paper formats, which are often susceptible to being misplaced. The lack of real-time updated centralized healthcare related information can lead to confusion regarding the benefits that are available under various plans and the responsibilities of the various cooperating parties (e.g., insurance provider, benefit plan administrator, covered party, and healthcare service provider) as it relates to healthcare reconciliation processing. In an illustrative embodiment, the disclosed system aggregates and consolidates data and information from various disparate resources into a store of healthcare related data 342.

In an illustrative implementation, healthcare identification and reconciliation platform 300 aggregates, stores, and provides access to various healthcare related data 342. Healthcare related data 342 may be any data that is relevant to a healthcare transaction. For example, in an illustrative implementation, healthcare related data 342 may comprise, in addition to a participating deductible amount on the document/screen, an actual amount that has been satisfied for a selected deductible period. For example, given a $2,500 deductible requirement for a benefit plan, and given that the covered party has already satisfied $250 of the deductible year to date, and the deductible is an annual deductible per person, healthcare identification and reconciliation platform 300 can calculate and store as healthcare related data 342 the remaining amount on the deductible. Healthcare identification and reconciliation platform 300 may be adapted to present this healthcare related data 342 (i.e., the participating user being responsible for the $2,250 of charges) to the participating user or other interested party.

In an illustrative implementation, healthcare identification and reconciliation platform 300 may be adapted to generate and communicate healthcare related data 342 comprising data representative of participating users' healthcare spending accounts. Such information may comprise, for example, account balances, restrictions on use of account funds, and warnings to use funds prior to account term (e.g., within a given tax year).

In an illustrative implementation, healthcare identification and reconciliation platform 300 may be adapted to generate, store, and communicate healthcare related data 342 that comprises data representative of indemnity plans or other specified benefit plans which might identify the amount of coverage that is available for healthcare services being rendered. In an illustrative implementation, a schedule of benefits for a plan or specific procedures can be generated and stored as healthcare related data 342. HIRP 300 may access such healthcare related data 342 in order to provide notice to cooperating parties of their responsibilities in the context of indemnification coverage (e.g., provide indemnifier's responsibilities and indemnitee's responsibilities).

In an illustrative implementation, healthcare identification and reconciliation platform 300 may be adapted to generate, store, and communicate healthcare related data 342 that comprises instructions for HMO's and other benefit plan providers to facilitate one or more selected plan requirements. For example, in an illustrative scenario, an HMO plan might cover lab work done at an on-site lab's, but will not cover lab work that is sent to an outside lab. Such information may be aggregated by HIRP 300 in healthcare related data 342. In response to a request for information, HIRP 300 may access such healthcare related data 342 in order to communicate an explanation of and instructions for such procedures. Providing access to such information will likely reduce confusion surrounding such requirements and eliminate unnecessary costs to the covered person.

In an illustrative implementation, healthcare identification and reconciliation platform 300 may comprise a multi level search engine, which may be, for example, comprised by healthcare identification and reconciliation engine 350. The search engine may operate to direct covered parties to specific groups of providers based on selected criteria and generate healthcare identification information/documents comprising information necessary to comply with the contractual requirements for such groups of providers. For example, in an illustrative scenario, in response to a request for information, a search engine may identify and communicate a first level of available providers that are covered by a plan (e.g, a primary PPO). The party accessing the HIRP may or may not select one of the providers identified by the search. If the first level is insufficient for the covered party's needs, a second level of providers covered by the plan is offered, and so on, until the covered party locates the general practitioner and/or specialist in group of providers (e.g., within a selected source of PPO providers).

In an illustrative implementation, healthcare identification and reconciliation platform 300 may be adapted to provide electronic links to specialty forms required to process various claims under a benefit plan. Such forms may be stored in and retrieved from, for example, healthcare related data 342. The links may be communicated, for example, as part of, or in connection with, communicating an electronic healthcare card/document. In an illustrative implementation, the forms can be made available through an electronic link on a display (not shown) of one or more client computing environments 320, 325, and/or 330 and may be customized for the specific services. Upon receipt, a cooperating party may activate the links which will cause the corresponding information (e.g., form) to be downloaded from HIRP 300 to the operator's computing system.

In another illustrative implementation, healthcare identification and reconciliation platform 300 may be adapted to aggregate, store, and communicate healthcare related data 342 that identifies specific vendor partners that have contracted with payors and that provides direction and steerage of covered parties to those partners. For example, a payor may have a contract with a national lab. In an illustrative implementation, HIRP 300 may be adapted to search healthcare related data 342 and place this information on the card/document/screen at the time of a patient's visit. Such information will remind cooperating parties of such relationships and in many instances will likely result in greater utilization of partner services and reduction of costs.

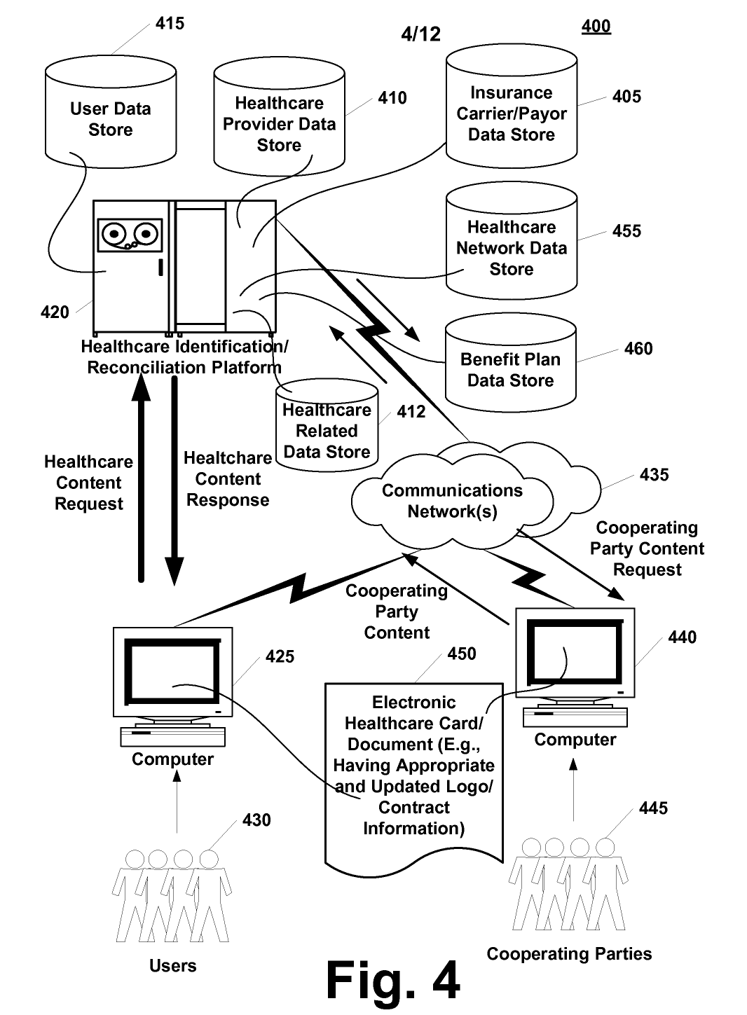

FIG. 4 shows a detailed illustrative implementation of exemplary healthcare identification and reconciliation environment 400. As is shown in FIG. 4, exemplary healthcare identification and reconciliation environment 400 comprises healthcare identification and reconciliation platform 420, user data store 415, healthcare provider data store 410, healthcare related information data store 412, insurance carrier/payor (or employer) data store 405, healthcare network data store 455, benefit plan data store 460, communications network 435, user computing environment 425, users 430, cooperating party computing environment 440, and cooperating parties 445. Additionally, as is shown in FIG. 4, healthcare identification and reconciliation environment 400 can comprise electronic healthcare card/document 450 which can be displayed, viewed, transmitted and/or printed from user computing environment 425 and/or cooperating party computing environment 440.

In an illustrative implementation, healthcare identification and reconciliation platform 420 can be electronically coupled to user computing environment 425 and cooperating party computing environment 440 via communications network 435. In the illustrative implementation, communications network can comprise fixed-wire and/or wireless intranets, extranets, and the Internet.

In an illustrative operation, users 430 can interact with an exemplary healthcare identification and reconciliation user interface (not shown) operating on user computing environment 425 to provide requests for healthcare identification and reconciliation information (e.g., electronic healthcare identification and reconciliation card/document) that are passed across communications network 435 to healthcare identification and reconciliation platform 420. In the illustrative operation healthcare identification and reconciliation platform 420 can process requests for healthcare identification and reconciliation information and cooperate with user data store 415, doctor healthcare provider data store 410, healthcare related information data store 412, healthcare network data store 455, benefit plan data store 460, and insurance carrier/payor data store 405 to generate electronic healthcare cards/documents for use by users 430 and cooperating parties 445.

In an illustrative implementation, user data store 415 can comprise, for example, data inputted to healthcare identification and reconciliation platform 420 by participating users 430 regarding the users' healthcare benefit plan. Such data can include but is not limited to insurance plan number data, member number data, group number data, and managed network data. In the illustrative implementation, healthcare provider data store 410 can comprise data representative of healthcare providers and their affiliations with various healthcare networks, fees, healthcare provider contact information, and healthcare provider requirements for accepting healthcare network coverage for a specific benefit plan. Healthcare related data store 412 may comprise any data that is relevant to a healthcare transaction. Healthcare related data store 412 may comprise, for example, any data that relates to healthcare and healthcare policies and procedures that may be useful to subscriber, healthcare provider, or other party, and may be created, for example, by aggregating and consolidated data from the other data sources. Insurance carrier/payor data store 405 can comprise data representative of various insurance carrier/payor responsibilities, practices, insurance carrier/payor contact information, eligibility requirements for members of a benefit plan, contractual requirements and other relevant information. Healthcare network data store 455 can comprise data such as contracts, fee schedules, plan designs, eligibility requirements, contact information, contractual obligations and other relevant information relevant to the healthcare network. Benefit plan data store 460 can comprise benefit plan component data, benefit plan eligibility requirements for members of the benefit plan, benefit plan differentials, benefit plan contractual requirements, benefit plan coverage, benefit plan payment limits, and other relevant data.

In the illustrative operation, responsive to the requests from users 430 for healthcare identification and reconciliation information, healthcare identification and reconciliation platform 420 can process the requests and correlate data from one or more cooperating data stores (e.g., user data store 415, healthcare provider data store 410, healthcare related data store 412, insurance carrier/payor data store 405, healthcare network data store 455, and benefit plan data store 460) to generate electronic healthcare card/document 450 having the most-up-to-date (e.g., current) healthcare identification and reconciliation information and healthcare related information 412 (for example, as described above in connection with FIG. 3) specific to the requesting user for communication to user computing environment 425 and/or cooperating party computing environment 440 through communications network 435. In the illustrative implementation, cooperating party computing environment 440 can comprise a healthcare provider computing environment and/or an insurance carrier/payor computing environment that can be used in the illustrative operation to display, view, transmit and/or print electronic healthcare card/document 450 for use in a variety of healthcare identification and reconciliation operations by cooperating parties 445 including, verifying the eligibility of a user for coverage under one or more benefit plans, providing contact information for use in payment reconciliation, and providing proof of one or more contractual obligations (e.g., placement of one or more logos) that need to be met for payment reconciliation between the user, healthcare provider, and/or insurance carrier/payor.

In an illustrative implementation, the generated healthcare identification and reconciliation information and healthcare related information/document 450 can be presented to a covered party and/or cooperating healthcare service provider via any suitable means including, for example, a screen of a computing device including, for example, PDA's, mobile phones, mobile e-mail device, etc.) or a printed form. The flexible electronic form factor and modality can increase availability and use of the generated healthcare identification and reconciliation information by cooperating healthcare service providers.

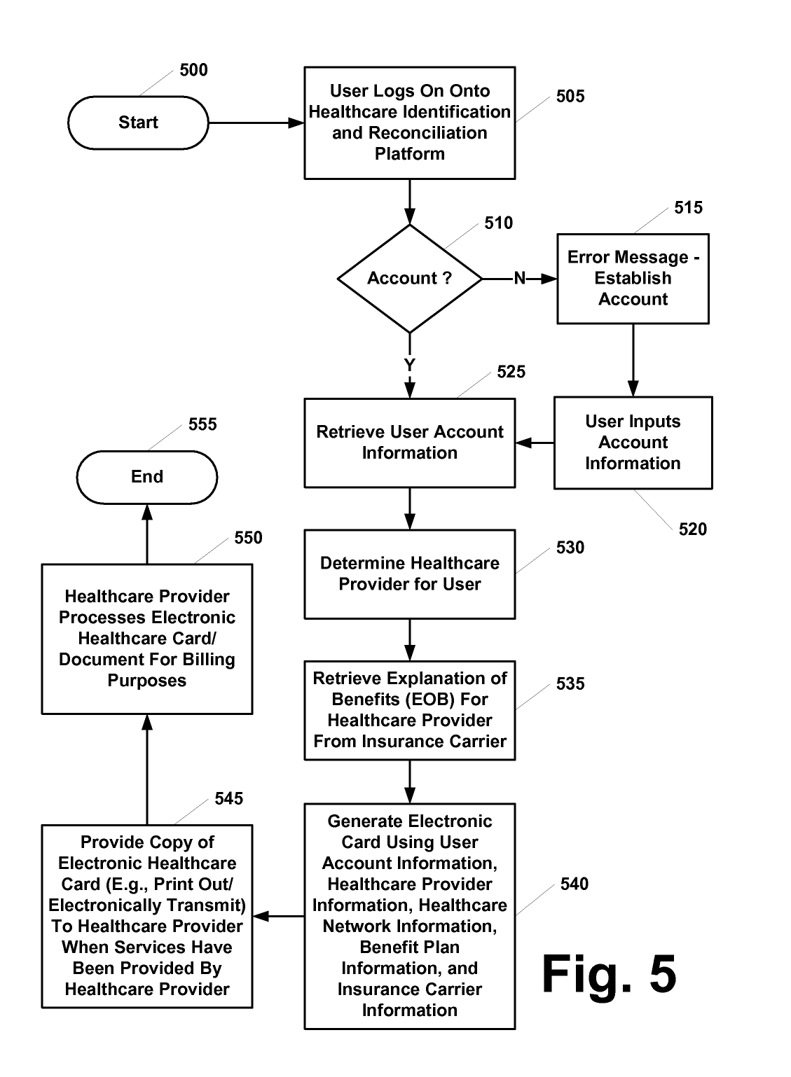

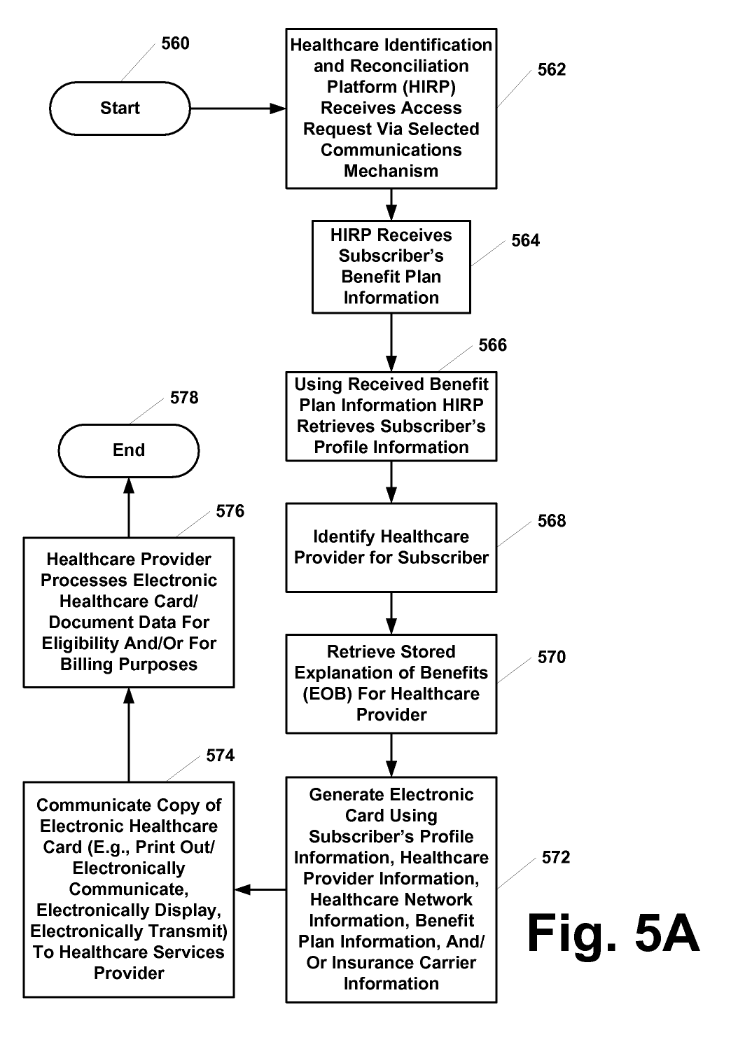

FIG. 5 shows exemplary processing performed when using an illustrative implementation of healthcare identification and reconciliation environment 400 of FIG. 4. As is shown, processing begins at block 500 and proceeds to block 505 where a user can log onto (e.g., using a secure logon mechanism—login id/password) the healthcare identification and reconciliation platform. From there a check is performed at block 510 to determine if the user has an account (e.g., or is eligible to view selected healthcare data) on the healthcare identification and reconciliation platform. If the check at block 510 indicates that the user does not have an account (or is ineligible), an error message is provided to the user at block 515. From there, the user is prompted to establish an account and the user can input account information at block 520. Processing then proceeds to block 525 and continues from there.

However, if at block 510 it is determined that the user has an account, processing proceeds to block 525 where the user account information is retrieved. From there, processing proceeds to block 530 where a healthcare provider/network is identified for the user (or by the user). As noted above in connection with FIG. 3, the healthcare identification and reconciliation platform may comprise a multi-level search engine that may direct parties to specific groups of providers depending upon the covered party's characteristics and search criteria. Once a healthcare provider/network is identified, a plan description can then be retrieved at block 535 which can be presented to the identified healthcare provider in the form of an electronic or hard copy healthcare card/document. The electronic healthcare card/document can then be generated at block 540 using the retrieved account information, healthcare provider information, healthcare network information, healthcare plan information and insurance carrier/payor information (e.g., EOB, EOD). In an illustrative embodiment, processing at block 540 may comprise accessing and processing healthcare related data 342 as described above in connection with FIG. 3. Processing then proceeds to block 545 where a copy (e.g., hard copy or electronic transmission) of the electronic healthcare card/document can be provided to the healthcare provider when services are provided by the healthcare provider. In an exemplary embodiment, the electronic healthcare card may be stored, for example with the user data, for later reference. The electronic healthcare card/document may comprise or may be supplemented with healthcare related data as described above in connection with FIG. 3. For example, the electronic healthcare card may be supplemented with links that may be used to access forms and related materials associated with the transaction. Responsive to receiving the copy of the electronic healthcare card/document, the healthcare provider can process the information provided on the electronic healthcare card/document as part of payment reconciliation (e.g., payment reconciliation with one or more insurance carriers/payors). Processing then terminates at block 555.

As noted above, the healthcare identification and reconciliation platform 430 may be accessed and used by any of numerous different cooperating parties to a healthcare transaction. The specific steps that a participating party may employ to access the system, and the particular processing that is performed, may vary depending upon the entity or person that is accessing the system as well as the particular information that the entity or person is seeking to obtain. FIG. 5A illustrates processing that may be performed using an illustrative implementation of healthcare identification and reconciliation environment 400 under a scenario that might be slightly different from that described in FIG. 5. As shown in FIG. 5, processing begins at block 560 and proceeds to block 562 where an exemplary health identification and reconciliation platform (HIRP) can receive a request for access. The request may be received, for example, from a subscriber (e.g., a patient) and/or from healthcare provider personnel such as, for example, an administrator, admitting nurse, billing specialist, receptionist, etc., in connection with an effort to confirm eligibility of a subscriber/patient and/or obtain subscriber benefit plan information. In an illustrative scenario, the subscriber/patient and/or healthcare provider personnel may seek to confirm eligibility and/or obtain subscriber benefit plan information, for example, at the time of, or in connection with, the patient's visit to the healthcare provider. The request for access may be received at the HIRP via any one or more communications mechanisms suitable for forwarding such a request including, but not limited to, a cooperating computing environment, a telephonic interface (with or without the assistance of customer service personnel), facsimile, e-mail, and/or short-message-service (SMS). The communication mechanism may comprise any of the Internet, a wireless telephonic network, a wireless communication network, fixed-wire communications network, and fixed-wire telephonic network

Referring to FIG. 5A, at block 564 the exemplary HIRP receives a subscriber's (e.g., patient's) benefit plan information via the one or more of the exemplary communications mechanisms. In an illustrative scenario, the subscriber's benefit plan information may have been entered and transmitted to the HIRP by, for example, the subscriber and/or healthcare provider personnel. The subscriber and/or healthcare provider personnel may have derived the transmitted benefit plan information from, for example, a previously generated electronic healthcare card, a non-electronically generated insurance ID card, or any other source.

At block 566, the exemplary HIRP uses the received benefit plan information to query its databases and retrieve the subscriber's (e.g., patient's) profile information. In an illustrative implementation, the subscriber's profile information can comprise any data including but not limited to the subscriber's benefit eligibility.

At block 568, the HIRP queries its databases to identify healthcare providers from whom the subscriber is eligible to receive services. In an illustrative embodiment, the identified healthcare providers may comprise those service providers from whom the particular subscriber qualifies to receive services as a result of the subscriber's particular benefit plan eligibility.

At block 570, the HIRP queries its databases to identify and retrieve information providing an explanation of benefits corresponding to the particular subscriber. In an illustrative embodiment, for example, the HIRP may retrieve information from its databases explaining the details of the benefits available to the subscriber under a particular benefit plan and the requirements for receiving services under the plan. In an illustrative embodiment, processing at block 570 may comprise accessing and processing healthcare related data 342 as described above in connection with FIG. 3.

Processing then proceeds to block 572 where the HIRP generates an electronic healthcare card using various data sources including but not limited to the subscriber's profile information, healthcare provider information, healthcare network information, benefit plan information, and/or insurance carrier information is generated. In an illustrative embodiment, the electronic healthcare card/document may comprise or may be supplemented with healthcare related data as described above in connection with FIG. 3. For example, the electronic healthcare card may be supplemented with links that may be used to access forms and related materials associated with the transaction.

At block 574, the HIRP communicates a copy of the generated electronic healthcare card to the requestor. In an illustrative scenario, the healthcare card information may be communicated to the subscriber and/or healthcare services provider personnel. The electronic healthcare card information may be communicated by any suitable mechanism including, for example, via telephone (electronically by automated interface and/or by a customer service representative cooperating with the exemplary HIRP), e-mail, facsimile, short messaging service, or any other suitable electronic or non-electronic communication mechanism. The received information may be displayed electronically and/or used to print in physical form. Those skilled in the art will appreciate that in addition to being communicated, the electronic healthcare card may be stored on the HIRP, for example with the user data, for later reference and analysis.

At step 576, the healthcare services provider can then process the generated and communicated electronic healthcare card as part of eligibility confirmation and/or billing processes. Processing of the request terminates at block 578.

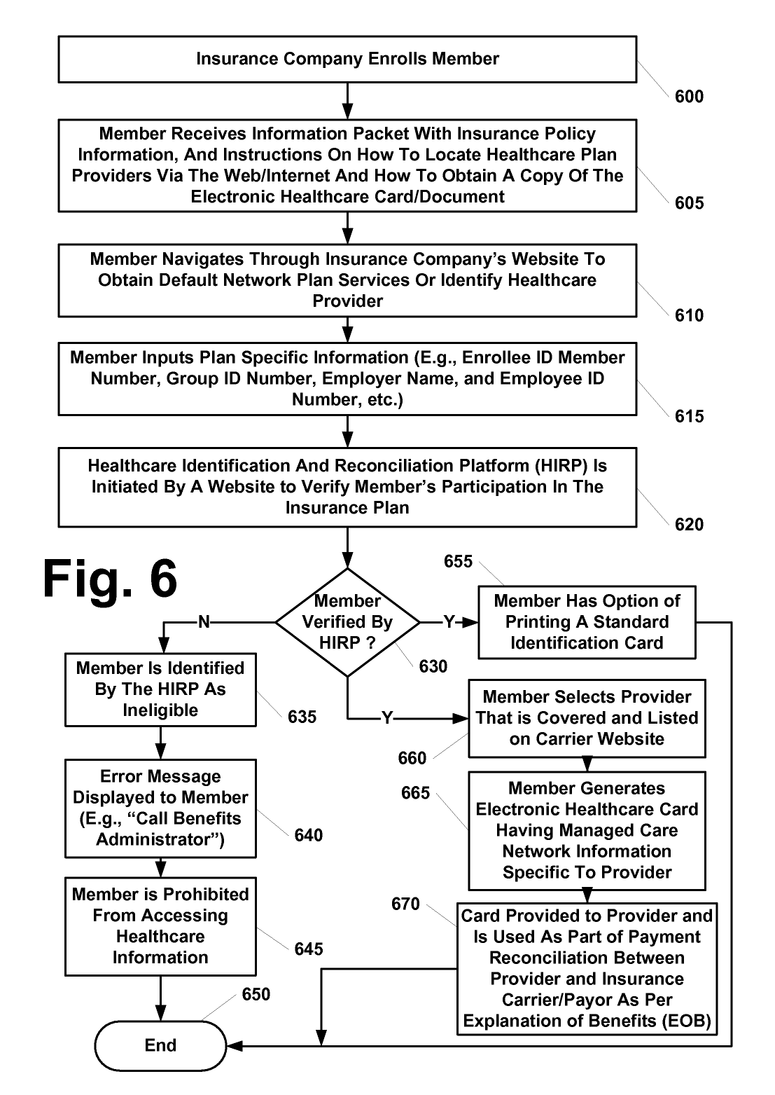

FIG. 6 shows other processing performed when using an illustrative implementation of healthcare identification and reconciliation environment of FIG. 4 in the context of processing health insurance policy claims. As is shown in FIG. 6, processing begins at block 600 where an insurance company enrolls a member. Processing then proceeds to block 605 where the member receives an information packet with insurance policy information, and instructions on how to locate healthcare plan providers using the world wide web/Internet, and how to print/transmit a copy of a generated electronic healthcare card/document. From there, processing proceeds to block 610 where a member can navigate through the insurance company's website to identify a healthcare provider. The member can be prompted by the insurance company's website to input insurance plan specific information (e.g., enrollee identification member number, group identification member number, employer name, and employee identification number) at block 615. The healthcare identification and reconciliation platform can then be initiated by the insurance provider's website to verify the member's participation in the insurance plan at block 620. This processing step can employ one or more predefined routines and/or algorithms (not shown) to correlate a plurality of data including but not limited to member data, insurance company data, and healthcare provider data, healthcare network data, and benefit plan data.

A check is then performed at block 630 to determine if the member has been verified by the healthcare identification and reconciliation platform. In this context, positive verification can be understood to mean that the member's insurance plan is current and the member is eligible to receive one or more insurance benefits. If the check at block 630 indicates that the member is not verified, processing proceeds to block 635 where the member is identified by the healthcare identification and reconciliation platform as ineligible. An error message is then displayed to the member on the insurance company's website to call a benefits administrator at block 640. The member is then prohibited from accessing selected healthcare information at block 645. Processing then terminates at block 650.

However, if the check at block 630 indicates that the member is verified, the member has two options. First, the member can use standard identification card (e.g., a healthcare card that is not specific to any particular healthcare provider or provides a default network) as at block 655. Processing then terminates at block 650. Second, the member can select a healthcare provider that is covered and listed on the insurance company's website at block 660. Upon selecting the desired healthcare provider, the member can then generate an electronic healthcare card (that is specific to the selected healthcare provider) at block 665 that can contain managed care network information specific to the selected healthcare provider. From there processing proceeds to block 670 where a copy (e.g., a printed copy, or an electronic copy) of the generated healthcare card is provided to the healthcare provider that can be used by the healthcare provider as part of payment reconciliation between the selected healthcare provider and the insurance company as per the insurance company's explanation of benefits (EOB). Processing then terminates at block 650.

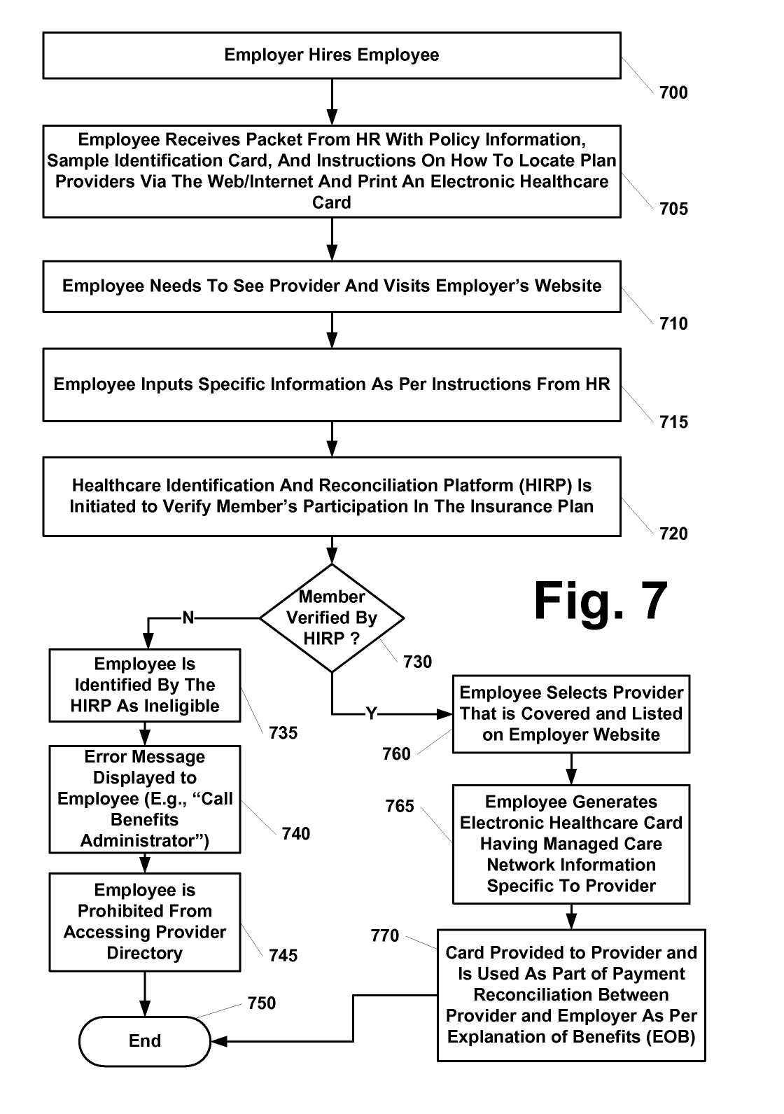

FIG. 7 shows other processing performed when using the illustrative implementation of healthcare identification and reconciliation environment of FIG. 4 from the perspective of an employer-provided benefits plan. As is shown in FIG. 7, processing begins at block 700 when an employer hires an employee. Processing then proceeds to block 705 where the employee receives an information packet with benefit plan information, and instructions on how to locate healthcare plan providers using the world wide web/Internet and how to print and/or transmit a copy of a generated electronic healthcare card. From there, processing proceeds to block 710 where an employee needs to see a provider and can navigate through the employer's website to identify a healthcare provider. The employee can be prompted by the employer's website as per human resources requirements to input plan specific information (e.g., enrollee identification member number, group identification member number, employer name, and employee identification number) at block 715. The healthcare identification and reconciliation platform can then be initiated by the employer's website to verify the member's participation in the benefit plan at block 720. This processing step can employ one or more predefined routines and/or algorithms (not shown) to correlate a plurality of data including but not limited to member data, insurance company data, healthcare network data, benefit plan data and healthcare provider data.

A check is then performed at block 730 to determine if the employee has been verified by the healthcare identification and reconciliation platform. In this context, verified can be understood to mean that the employee's benefit plan is current and that the employee is eligible to receive one or more plan benefits. If the check at block 730 indicates that the employee is not verified, processing proceeds to block 735 where the employee is identified by the healthcare identification and reconciliation platform as ineligible. An error message is then displayed to the employee on the employer's website to call a benefits administrator at block 740. The employee is then prohibited from accessing any additional information such as a healthcare provider directory at block 745. Processing then terminates at block 750.

However, if the check at block 730 indicates that the employee is verified, the employee can select a healthcare provider that is covered and listed on the employer's website at block 760. Upon selecting the desired healthcare provider, the employee can then generate an electronic healthcare card (that is specific to the selected healthcare provider and/or network) at block 765 that can contain managed care network information specific to the selected healthcare provider. From there processing proceeds to block 770 where a copy (e.g., a printed copy, or an electronic copy) of the generated healthcare card is provided to the healthcare provider that can be used by the healthcare provider as part of payment reconciliation between the selected healthcare provider and the insurance company as per the insurance company's explanation of benefits, explanation of discount (EOB, EOD). Processing then terminates at block 750.

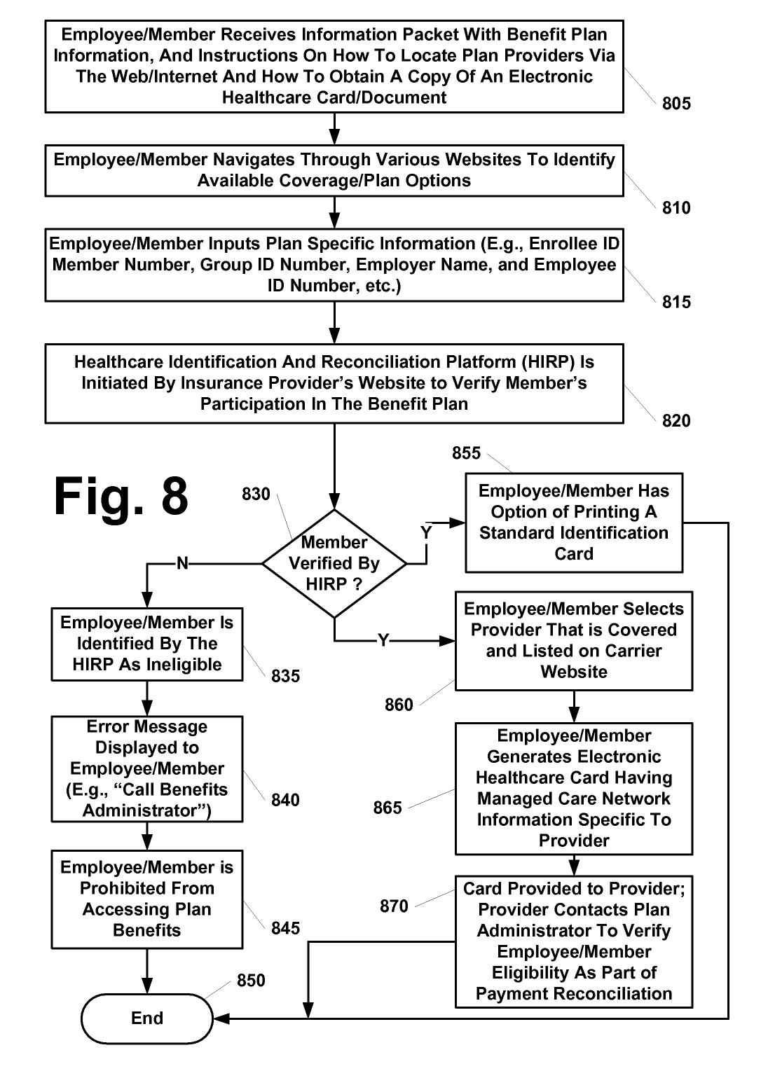

FIG. 8 shows other processing performed when using an illustrative implementation of healthcare identification and reconciliation environment of FIG. 4 in the context of processing medical benefit plan administration for employees and/or members. As is shown in FIG. 8, processing begins at block 805 where the employee/member receives an information packet with benefit plan information, and instructions on how to access and/or locate healthcare plan providers using the world wide web/Internet and how to generate a copy of a electronic healthcare card/document. From there, processing proceeds to block 810 where an employee/member can navigate through various insurance company's/payors and/or plan websites to identify various options. The employee/member can be prompted by the website to input benefit plan specific information (e.g., enrollee identification member number, group identification member number, employer name, and employee identification number) at block 815. The healthcare identification and reconciliation platform can then be initiated by the website to verify the employee's/member's participation in the insurance plan at block 820. This processing step can employ one or more predefined routines and/or algorithms (not shown) to correlate a plurality of data including but not limited to employee/member data, insurance company data, healthcare network data, benefit plan data and healthcare provider data.

A check is then performed at block 830 to determine if the employee/member has been verified by the healthcare identification and reconciliation platform. In this context, verified can be understood to mean that the employee's/member's benefit plan is current and that the employee/member is eligible to receive one or more insurance benefits. If the check at block 830 indicates that the employee/member is not verified, processing proceeds to block 835 where the employee/member is identified by the healthcare identification and reconciliation platform as ineligible. An error message is then displayed to the employee/member on the insurance company's website to call a benefits administrator at block 840. The employee/member is then prohibited from accessing plan benefits block 845. Processing then terminates at block 850.

However, if the check at block 830 indicates that the employee/member is verified, the employee/member has two options. First, the employee/member has the option of generating and/or printing out a standard identification card (e.g., a healthcare card that is inclusive of a standard set of selected options and not specific to any particular healthcare provider) at block 855. Processing then terminates at block 850. Second, the employee/member can select a healthcare provider that is covered and listed as part of the insurance/payor benefit plan at block 860. Upon selecting the desired healthcare provider, the employee/member can then generate an electronic healthcare card/document (that is specific to the selected healthcare provider) at block 865 that can contain managed care network information specific to the selected healthcare provider along with the related contractual requirements associated with the healthcare network and/or benefit plan. From there processing proceeds to block 870 where a copy (e.g., a printed copy, or an electronic copy) of the generated healthcare card is provided to the user or healthcare provider that can be utilized by the healthcare provider as part of payment reconciliation between the selected healthcare provider and the insurance company/payor as per the provided explanation of benefits (EOB). In an illustrative implementation, the healthcare provider can use the information provided on the electronic healthcare card/document to identify the fee schedule and/or network and plan affiliation as well identify contact information for use in contacting the plan administrator to verify the member's information. Processing then terminates at block 850.

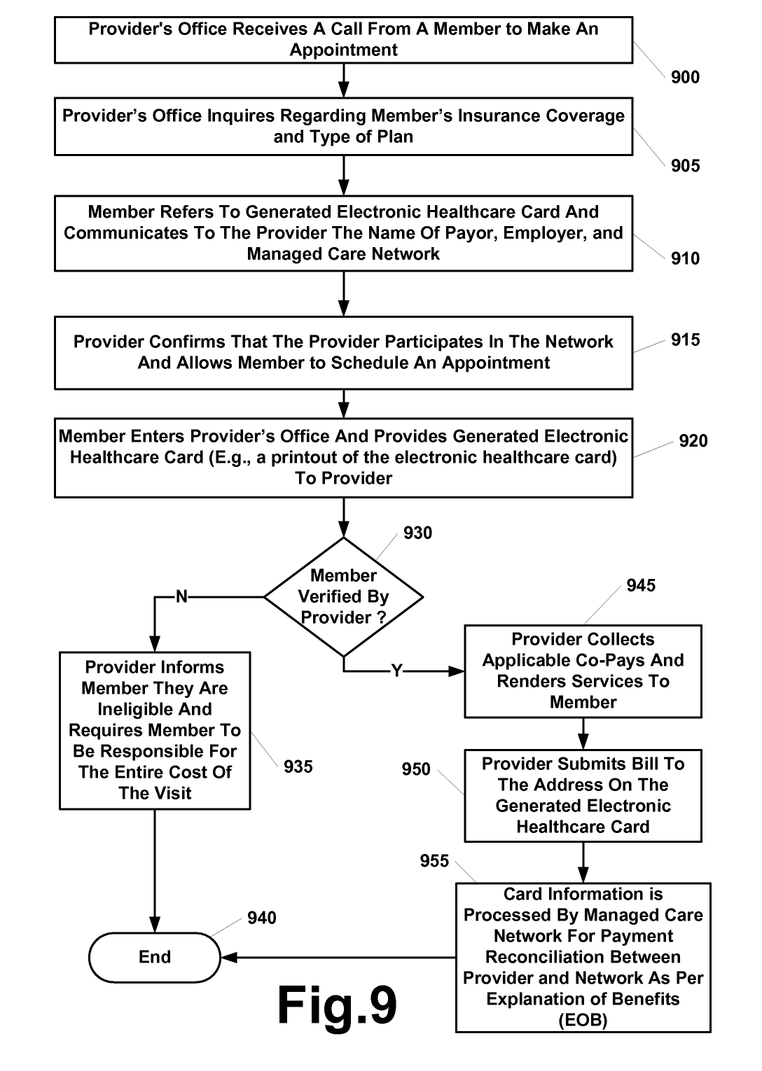

FIG. 9 shows other processing performed when using an illustrative implementation of healthcare identification and reconciliation environment of FIG. 4 in the context of the processing performed by a healthcare provider when handling electronic healthcare card information. As is shown in FIG. 9, processing begins at block 900 where a healthcare provider's office receives a call from a member to make an appointment. The healthcare provider's office then inquires at block 905 regarding the member's insurance coverage and type of plan. Responsive to such inquiry, the member can then refer to a generated electronic healthcare card (i.e., generated by the healthcare identification and reconciliation platform) and communicate to the healthcare provider at block 910 the information requested, such as the name of the payor, employer, and/or managed care network information. The healthcare provider can then confirm that the communicated information and allows the member to schedule an appointment. The member can then provide a copy of the generated electronic healthcare card to the healthcare provider at their scheduled appointment at block 920. The healthcare provider can then perform a check to determine if the member is still eligible to receive discounted services from the healthcare provider at block 930.

If the check at block 930 indicates that the member is no longer eligible (e.g., member's plan was changed, the healthcare provider left the member's network, etc.), the provider informs the member that the member is ineligible and requires the member to be responsible for the entire cost of the visit. Processing then terminates at block 940. However, if at block 930, the member is verified, processing proceeds to block 945 where the healthcare provider collects applicable co-pays and renders services to the member. The healthcare provider can then submit a bill to the address on the generated electronic healthcare card at block 950. The card information can then be processed by the managed care network at block 955 for payment reconciliation between the provider and the network as per the insurance carrier's/payor's EOB. Processing then terminates at block 940.

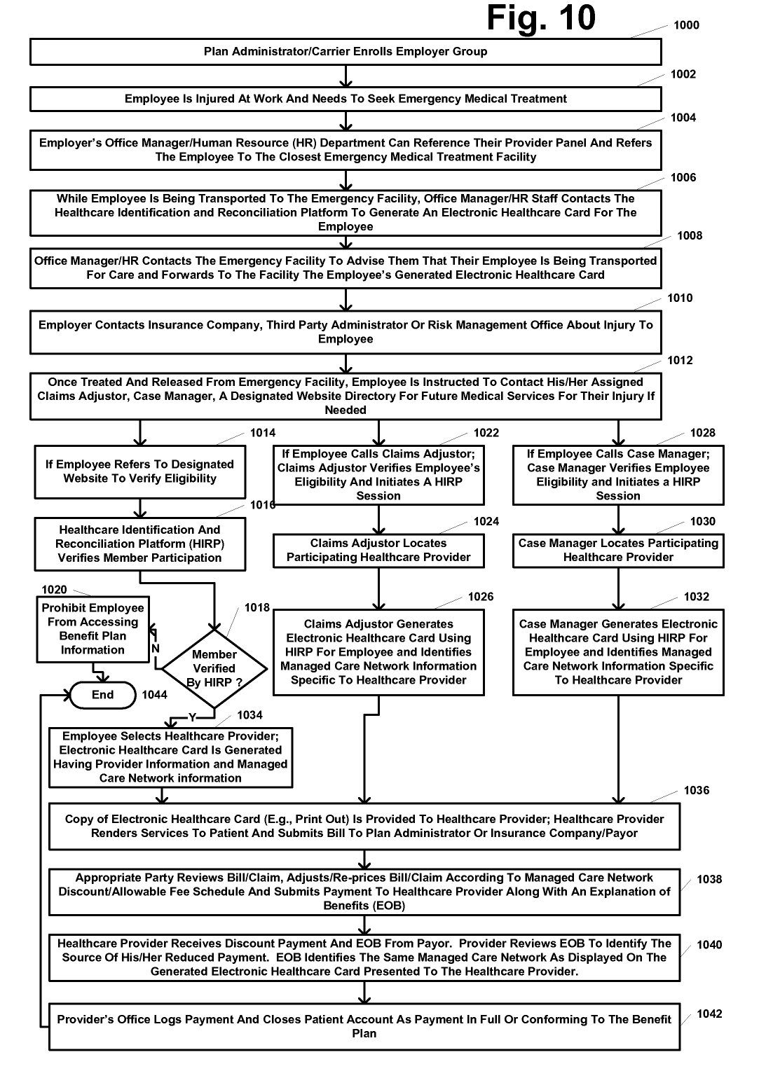

FIG. 10 shows other processing performed when using an illustrative implementation of the healthcare identification and reconciliation environment of FIG. 4 in the context of processing workman's compensation benefits. As is shown in FIG. 10, processing begins at block 1000 where a plan administrator and/or insurance company enrolls an employer group. From there, processing proceeds to block 1002 for an employee injured at work who needs to seek emergency medical treatment. The employer's office manager/human resource department can reference their healthcare provider panel and refer the employee to the closest emergency medical treatment facility at block 1004. While the employee is being transported to the emergency facility, the office manager/human resources staff can refer to the healthcare identification and reconciliation platform to generate an electronic healthcare card/document for the employee at block 1006. From there, processing proceeds to block 1008 where the office manager/human resources personnel contacts the emergency facility to advise them that their employee is being transported for care and forwards to the facility the employee's generated electronic healthcare card/document. The employer contacts the insurance company, third party administrator or risk management office about the employee's injury at block 1010. Once treated and released from the emergency facility, the employee is instructed at block 1012 to contact his/her assigned claims adjustor, case manager, or a designated website directory to identify a healthcare provider for future related medical service if needed.

If employee refers to the designated website, processing proceeds to block 1014 where the employee verifies their eligibility for coverage by the insurance company/payor. Processing then proceeds to block 1016 where a session on the healthcare identification and reconciliation platform is initiated to identify benefit plan related options. A check is then performed at block 1018 to determine if the healthcare identification and reconciliation platform has verified the employee. If the check at block 1018 indicates that the employee is not verified, processing proceeds to block 1020 where the employee is prohibited from accessing further healthcare information that can be found on the designated website. Processing then terminates at block 1044.

However if the check at block 1018 indicates that the healthcare identification and reconciliation platform has verified the member, processing proceeds to block 1034 where the employee selects a healthcare provider from a healthcare provider directory and an electronic healthcare card/document is generated having healthcare provider information and managed care network information along with other contractual specifications required as part of the administration of the given benefit plan. A copy of the generated electronic healthcare card/document is provided to the healthcare provider during the employee's visit at block 1036. Additionally, at block 1036 the healthcare provider renders services to the patient and submits a bill to the insurance company/payor, as instructed by the insurance company/payor, for payment. Responsive to the submission of the bill, at block 1038, the insurance company/payor or plan administrator reviews the bill/claim and re-prices/adjusts the bill/claim according to a selected managed care network discount and/or fee schedule that is allowable and submits the payment to the healthcare provider along with an explanation of benefits (EOB). The healthcare provider receives the discount payment and EOB from the insurance company/payor at block 1040. The healthcare provider reviews the EOB to identify the adjustments and the source of the adjusted payment. The EOB identifies the information required by contract such as a logo representative of managed care network as displayed on the generated electronic healthcare card presented to the healthcare provider. From there processing proceeds to block 1042 where the healthcare provider's office logs payment and closes the patient account (e.g., employee account) as payment in full or in conformity with the plan design. Processing then terminates at block 1044.

In the instance where the employee contacts the claim adjustor as per the recommendation of block 1012, at block 1022, the employee contacts the claim adjustor and the claims adjustor verifies the employee's eligibility and initiates a session on the healthcare identification and reconciliation platform. Using the information provided by the healthcare identification and reconciliation platform, the claims adjustor can locate a participating healthcare provider at block 1024. The claims adjustor can then, at block 1026, generate an electronic healthcare card/document using the healthcare identification and reconciliation platform for the employee and identifies the managed care network information specific to the healthcare provider for inclusion on the generated electronic healthcare card/document. Processing proceeds to block 1036 and continues from there.

In the instance where the employee contacts the case manager as per the recommendation of block 1012, at block 1028, the employee contacts the case manager and the case manager verifies the employee's eligibility and initiates a session on the healthcare identification and reconciliation platform. Using the information provided by the healthcare identification and reconciliation platform, the case manager can locate a participating healthcare provider at block 1030. The case manager can then, at block 1032, generate an electronic healthcare card/document using the healthcare identification and reconciliation platform for the employee and identifies the managed care network information specific to the healthcare provider for inclusion on the generated electronic healthcare card/document. Processing proceeds to block 1036 and continues from there.

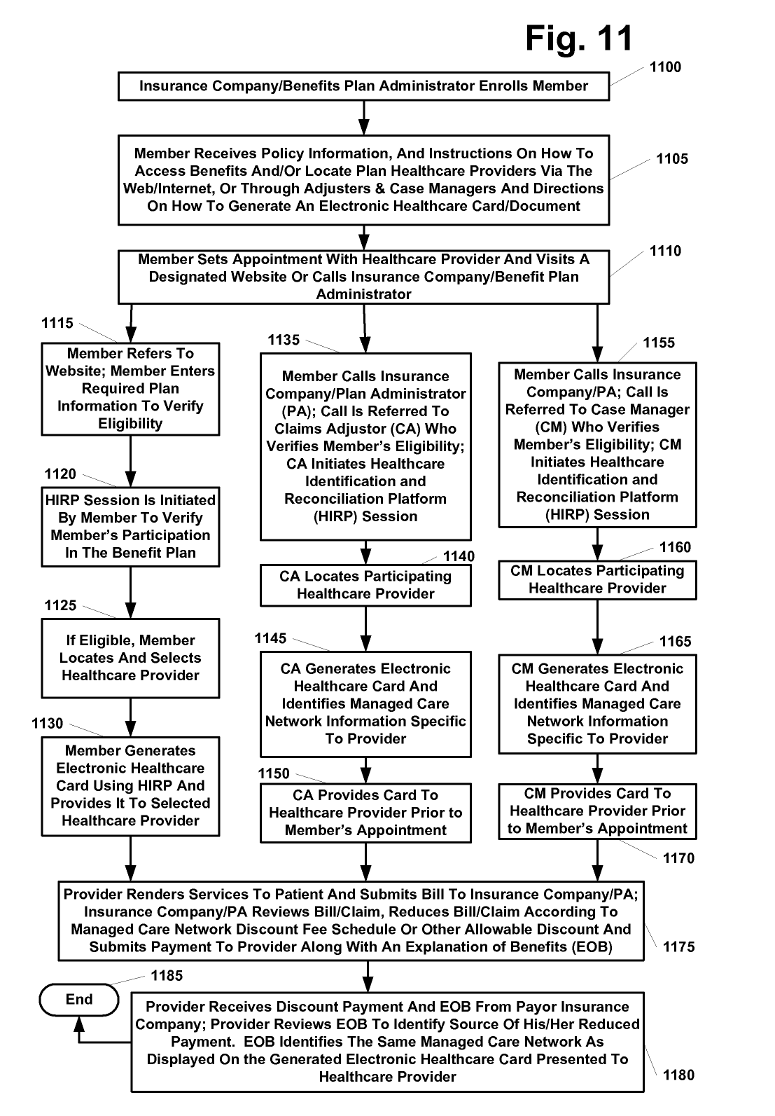

FIG. 11 shows other processing performed when using an illustrative implementation of healthcare identification and reconciliation environment of FIG. 4 in the context of processing occupational health and non-subscriber benefits. As is shown in FIG. 11, processing begins at block 1100 where an insurance company/plan administrator enrolls a member. Processing then proceeds to block 1105 where the member receives insurance policy information, and instructions on how to access plan benefits and/or locate healthcare plan providers via the web/Internet, or through claims adjustors, case managers and directions on how to generate an electronic healthcare card/document. The member can then make an appointment to visit a selected healthcare provider and can navigate to the designated website or call the plan administrator/insurance company at block 1110.

If the member refers to the designated website, the member can be prompted to input required plan information to verify eligibility at block 1115. A session is then initiated at block 1120 on the healthcare identification and reconciliation platform for the member by the designated website to verify the member's participation in the benefit plan. Such verification can be realized by correlating in real time updated insurance company information, member information, benefit plan information, healthcare network information and healthcare provider information. If eligible, the member can locate and select their healthcare provider at block 1125. The member can then generate an electronic healthcare card/document using the healthcare identification and reconciliation platform and can provide it to the healthcare provider upon their scheduled visit at block 1130. The healthcare provider then can render services to the member and submit a bill to the designated location such as the plan administrator/insurance company at block 1175 for services rendered. Additionally, at block 1175, the plan administrator/insurance company can review the bill/claim and reduce the bill/claim according to managed care network discount/fee schedules/other allowable limits and submit payment to the healthcare provider along with an explanation of benefits (EOB). From there, processing can proceeds to block 1180 where the provider receives the discount payment and EOB from the payor/insurance company, reviews the EOB to identify the source of the reduced payment. The EOB identifies the same managed care network information as displayed on the generated electronic healthcare card as the source of the discount. Processing then terminates at block 11185.

If the member calls the plan administrator/insurance company as prescribed at block 1110, the call can be referred to a claims adjustor who verifies the member's eligibility under the benefit plan. From there, the claims adjustor locates a participating healthcare provider at block 1140. The claims adjustor can then generate an electronic healthcare card/document using the healthcare identification and reconciliation platform which can operate to identify the contractual requirements such as managed care network information specific to the selected healthcare provider at block 1145. The claims adjustor can then provide the generated electronic healthcare card/document to the selected provider (e.g., electronically/fax/mail) prior to the member's appointment. Processing then proceeds to block 1175 and continues from there.

If the member calls the plan administrator/insurance company as prescribed at block 1110, the call can be referred to a case manager who verifies the member's eligibility under the benefit plan. From there, the case manager locates a participating healthcare provider at block 1160. The case manager can then generate an electronic healthcare card/document using the healthcare identification and reconciliation platform which can operate to identify the contractual requirements such as managed care network information specific to the selected healthcare provider at block 1165. The case manager can then provide the generated electronic healthcare card/document to the selected provider (e.g., electronically/fax/mail) prior to the member's appointment. Processing then proceeds to block 1175 and continues from there.

It is understood that the herein described systems and methods are susceptible to various modifications and alternative constructions. There is no intention to limit the herein described systems and methods to the specific constructions described herein. On the contrary, the herein described systems and methods are intended to cover all modifications, alternative constructions, and equivalents falling within the scope and spirit of the herein described systems and methods.

It should also be noted that the herein described systems and methods can be implemented in a variety of electronic environments (including both non-wireless and wireless computer environments), partial computing environments, and real world environments. The various techniques described herein may be implemented in hardware or software, or a combination of both. Preferably, the techniques are implemented in computing environments maintaining programmable computers that include a computer network, processor, servers, a storage medium readable by the processor (including volatile and non-volatile memory and/or storage elements), at least one input device, and at least one output device. Computing hardware logic cooperating with various instructions sets are applied to data to perform the functions described above and to generate output information. The output information is applied to one or more output devices. Programs used by the exemplary computing hardware may be preferably implemented in various programming languages, including high level procedural or object oriented programming language to communicate with a computer system. Illustratively the herein described apparatus and methods may be implemented in assembly or machine language, if desired. In any case, the language may be a compiled or interpreted language. Each such computer program is preferably stored on a storage medium or device (e.g., ROM or magnetic disk) that is readable by a general or special purpose programmable computer for configuring and operating the computer when the storage medium or device is read by the computer to perform the procedures described above. The apparatus may also be considered to be implemented as a computer-readable storage medium, configured with a computer program, where the storage medium so configured causes a computer to operate in a specific and predefined manner.

Although exemplary implementations of the herein described systems and methods have been described in detail above, those skilled in the art will readily appreciate that many additional modifications are possible in the exemplary embodiments without materially departing from the novel teachings and advantages of the herein described systems and methods. Accordingly, these and all such modifications are intended to be included within the scope of the herein described systems and methods. The herein described systems and methods may be better defined by the following exemplary claims.

What is claimed is:

1. A system for generating healthcare information and reconciliation comprising:
a data store comprising patient data, healthcare provider data, insurance carrier data, healthcare network data, and benefit plan data;
a computing processor communicatively coupled with the data store; and
computing memory communicatively coupled with the computing processor, the computing memory having instructions stored therein which when executed by the computing processor cause the processor to perform the following method:
receiving data representative of a subscriber's benefit plan information via an electronic communication mechanism;
correlating the received data against one or more of healthcare provider data, healthcare network data, benefit plan data and insurance carrier data; and
generating an electronic healthcare card having information thereon that is representative of current contractual obligations existing between cooperating parties of a healthcare system.

2. The system as recited in claim 1 wherein the cooperating parties of a healthcare system comprise any of a patient, a healthcare provider, an insurance carrier, and employer, a claims adjustor, a case manager, a healthcare network, and a payor.

3. The system as recited in claim 1 wherein the electronic healthcare card information comprises managed network healthcare information specific to a selected healthcare provider.

4. The system as recited in claim 3 wherein the healthcare card information comprises one or more selected logos to satisfy one or more contractual obligations existing between a healthcare provider and an insurance carrier or payor.

5. The system as recited in claim 1, wherein receiving data representative of a subscriber's benefit plan information via an electronic communication mechanism comprises receiving data via one or more of the Internet, a wireless telephonic network, a wireless communication network, fixed-wire communications network, and fixed-wire telephonic network.

6. The system as recited in claim 1, wherein receiving data representative of a subscriber's benefit plan information via an electronic communication mechanism comprises receiving data communicated by the subscriber.

7. The system as recited in claim 1, wherein receiving data representative of a subscriber's benefit plan information via an electronic communication mechanism comprises receiving data communicated by healthcare service provider personnel.

8. The system as recited in claim 7 further comprising communicating the electronic healthcare card to healthcare service provider personnel.

9. The system as recited in claim 1 wherein receiving data representative of a subscriber's benefit plan information comprises receiving any of user benefit plan data, healthcare provider data, employer data, claims adjustor data, case manager data, healthcare network data, and insurance carrier/payor data.

10. The system as recited in claim 7 wherein communicating the electronic healthcare card comprises communicating information via at least one of phone, facsimile, e-mail, or short messaging service.

11. A computer-implemented interactive method for generating current healthcare identification and reconciliation information, comprising:
receiving data representative of a subscriber's benefit plan information via an electronic communication mechanism;
correlating the received data against one or more of healthcare provider data, healthcare network data, benefit plan data and insurance carrier/payor data to produce verified healthcare information for the participating user;
generating an electronic healthcare card/document having verified healthcare information representative of one or more current relationships between cooperating parties comprising any of healthcare providers, benefit plan administrators, healthcare networks, insurance carriers/payors, and participating users.

12. The method as recited in claim 11 further comprising generating verified healthcare information comprising one or more selected logos representative of one or more current relationships between cooperating parties comprising any of a healthcare provider, benefit plan administrator, healthcare network and an insurance carrier/payor.

13. The method as recited in claim 12 further comprising providing an electronic healthcare card/document for delivery to healthcare provider in accordance with contractual requirements.

14. The method as recited in claim 13 further comprising reconciling payment for services rendered by a healthcare provider using the generated electronic healthcare card/document.

15. A computer-readable medium that contains instructions which, when executed by a processor, causes the processor to perform an interactive method for generating healthcare identification and reconciliation information comprising the steps of:
receiving data representative of a subscriber's current benefit plan information via an electronic communication mechanism, the electronic communication mechanism comprising at least one of: the Internet, a wireless telephonic network, a wireless communication network, fixed-wire communications network, or fixed-wire telephonic network;
correlating the received data against one or more data comprising any of healthcare provider data, healthcare network data, benefit plan data and insurance carrier/payor data to produce verified healthcare information for the subscriber;
generating an electronic healthcare card/document having verified healthcare information representative of one or more current relationships between cooperating parties comprising any of healthcare providers, benefit plan administrators, healthcare networks, insurance carriers/payors, and participating users.

16. A method to generate an electronic healthcare card comprising:
receiving data representative of a benefit plan;
comparing the received benefit plan data with data in a cooperating data store to determine eligibility requirements for the benefit plan;
upon satisfying eligibility requirements retrieving current data from a cooperating data store representative of healthcare providers that are covered by the benefit plan; and
generating an electronic healthcare card/document using the received data representative of the benefit plan and the retrieved healthcare provider information such that the electronic healthcare card/document has current information specific to one or more of the covered healthcare providers.

17. The method as recited in claim 16 further comprising generating an electronic healthcare card/document having information specific to one or more healthcare providers such that the electronic healthcare card/document satisfies one or more current contractual obligations that exist between the one or more healthcare providers and the benefit provider.

18. A system for generating healthcare information and reconciliation comprising:
a data store comprising patient data, healthcare provider data, insurance carrier data, healthcare network data, healthcare related data, and benefit plan data;
a computing processor communicatively coupled with the data store; and
computing memory communicatively coupled with the computing processor, the computing memory having instructions stored therein which when executed by the computing processor cause the processor to perform the following method:
receiving data identifying a covered party via an electronic communication mechanism;
in response to the request, generating an electronic healthcare card comprising: information representative of current contractual obligations existing between cooperating parties of a healthcare system, and healthcare related data; and
communicating the electronic healthcare card.

19. The system as recited in claim 18, wherein the healthcare related data comprises deductible information.

20. The system as recited in claim 18, wherein the healthcare related data comprises data representative of healthcare spending accounts.

21. The system as recited in claim 18, wherein the healthcare related data comprises data representative of indemnity plans.

22. The system as recited in claim 18, wherein the healthcare related data comprises data representative of instructions for processing payment of healthcare services by parties comprising health maintenance organizations, benefit plan operators, and personal plan option operators.

23. The system as recited in claim 18, further comprising instructions stored in the memory which when executed by the computing processor cause the processor to perform searches for healthcare service providers satisfying particular criteria.

24. The system as recited in claim 18, wherein communicating the electronic healthcare card comprises communicating one or more electronic links to one or more forms for use as part of healthcare reconciliation processing.

25. The system as recited in claim 18, further comprising instructions stored in the memory which when executed by the computing processor cause the processor to communicate one or more links to one or more forms, the forms comprising information corresponding to information on the electronic healthcare card.

26. The system as recited in claim 18, wherein the data store comprises partner data representative of at least one vendor partner capable of offering services to a covered party, and
wherein generating an electronic healthcare card comprises highlighting the at least one vendor partner on the electronic healthcare card.

27. The system as recited in claim 18, further comprising instructions stored in the memory which when executed by the computing processor cause the processor to store a generated electronic healthcare card for subsequent use.
